(19) United States
(12) Patent Application Publication
(10) Pub. No.: US 2005/0288784 A1
(43) Pub. Date: Dec. 29, 2005

(54) Bifocal intraocular telescope for low vision correction

(76) Inventor: Gholam A. Peyman, New Orleans, LA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/873,495

(22) Filed: Jun. 23, 2004

(51) Int. Cl. A61F 2/16

(52) U.S. Cl. 623/6.35; 623/6.34; 623/6.36

(57) ABSTRACT

An intraocular lens implant has a telescope portion and a transparent peripheral portion coupled to the outside of the telescope portion. The telescope portion has a converging lens and a diverging lens to form a Galilean telescope. The telescope portion provides magnified vision for the central field of vision. The peripheral portion of the implant is adapted to correct for refractive errors and allows for unrestricted peripheral vision.

### CROSS REFERENCE TO RELATED APPLICATIONS

This application is related to U.S. application Ser. No. 10/455,788, filed Jun. 6, 2003, entitled “TELEDIOPTIC LENS SYSTEM AND METHOD FOR USING THE SAME,” and U.S. application Ser. No. 10/600,371, filed Jun. 23, 2003, entitled “TELEDIOPTIC LENS SYSTEM AND METHOD FOR USING THE SAME.” The entire contents of both of these applications are incorporated herein by reference.

### FIELD OF THE INVENTION

The present invention generally relates to an intraocular lens for implantation in an eye. More specifically, the present invention relates to an intraocular telescope for correction of low vision caused by macular degeneration.

### BACKGROUND OF THE INVENTION

Macular degeneration has become one of the leading causes of blindness in adults. This disease affects the central retinal area known as the macula. The macula is responsible for acute vision—i.e., vision for such things as driving or reading a newspaper. Macular degeneration can lead to a gradual or sudden loss of vision to the level of 20/200 or less. Commonly, loss of vision only affects the central macular area of about 0.25 to 4 square millimeters, and does not usually progress beyond this area, thereby leaving 95-99% of the retina unaffected. Thus, reading and driving vision can be lost, while peripheral vision remains intact. This condition is often referred to as low vision.

Most cases of macular degeneration are untreatable, although laser photocoagulation has been successful in certain instances. Telescopic systems that attach to eye glasses also have been used for many years to improve vision in patients with macular degeneration. These systems, which work by increasing the retinal image of a given object, have not been very successful because they restrict the visual field to about 11° so that normal activity is not possible. They are also large and bulky. Attempts have been made to increase the visual field by putting part of the telescope within the eye. A Galilean telescope is useful for this purpose and consists of a converging objective lens and a diverging ocular lens, which together produce a telescopic effect.

U.S. Pat. Nos. 4,666,446 and 4,581,031, both to Koziol and Peyman, and both of which are incorporated by reference herein, each disclose intraocular lenses which are implanted in the eye in place of the natural lens to redirect the rays of light to minimize the adverse affect on vision caused by the macular degeneration of the eye. For example, U.S. Pat. No. 4,666,446 discloses an intraocular lens comprising a first portion including a diverging lens and a second portion including a converging lens. The converging lens provides the eye with substantially the same focusing ability of the natural lens prior to implantation of the intraocular lens. Thus, the eye will have decreased visual acuity due to the macular degeneration, but will also have unrestricted peripheral vision. The diverging lens, on the other hand, when combined with a converging lens positioned outside of the eye (e.g., a spectacle lens), provides a magnified image with increased visual acuity but a restricted visual field. Therefore, this type of intraocular lens creates a teledioptic lens system, which provides the patient with the choice of unmagnified but peripherally unrestricted vision or magnified but peripherally restricted vision.

U.S. Pat. No. 6,197,057 to Peyman and Koziol, the entire contents of which are herein incorporated by reference, relates to a lens system that combines a high plus lens with a plus and minus intraocular lens (IOL), so that the lens system works in a manner similar to a Galilean telescope. Generally the high plus lens is outside the eye (i.e., in glasses or spectacles or in a contact lens) and the plus and minus lens is an IOL that replaces or works in conjunction with the natural lens of the patient (See FIGS. 1 and 2).

U.S. Pat. Nos. 4,074,368 and 6,596,026 B1, the entire contents of which are herein incorporated by reference, both disclose telescopic implants for implantation within an eye. These implants are designed to replace the natural lens in the eye with a telescope. They are rigid devices requiring a large incision in the eye to implant.

Although all of these systems are beneficial to patients with macular degeneration, a continuing need exists for an intraocular implant that can correct for low vision in the eye.

### SUMMARY OF THE INVENTION

An object of the present invention is to provide a telescopic intraocular lens for implantation in an eye to correct for macular degeneration.

Another object of the present invention is to provide an intraocular lens for implantation in an eye that provides both unmagnified and peripherally unrestricted vision and magnified and peripherally restricted vision to correct for macular degeneration.

A further object of the present invention is to provide an intraocular lens for implantation in an eye to create a lens system that redirects rays of light away from a diseased portion of the retina in the eye and focuses those rays onto an un-diseased area of the eye.

Yet another object of the present invention is to provide an intraocular lens implant that is small enough to be implantable through a relatively small incision in the eye, and can provide bifocal correction to the eye.

These and other objects of the invention are achieved by an intraocular lens implant having a telescope portion and a peripheral portion coupled to the outside of the telescope portion. The telescope portion has a converging lens and a diverging lens to form a Galilean telescope, providing magnified vision for reading, driving, and other activities requiring acute vision. The peripheral portion is optically transparent, providing unmagnified peripheral vision but can have refractive powers to provide bifocal vision correction to the eye in conjunction with the telescope portion. A set of haptics is attached to the peripheral portion for fixating the peripheral portion in an eye.

Other objects, advantages, and salient features of the present invention will become apparent from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 is a cross-sectional view in side elevation of a human eye with an intraocular implant according to a first embodiment of the present invention;

FIG. 2 is an enlarged cross-sectional view in side elevation of the telescope portion of the implant shown in FIG. 1 having a plus and a minus lens therein;

FIG. 3 is a top plan view of the intraocular implant shown in FIG. 1 prior to implantation;

FIG. 4 is a side elevational view of the intraocular implant shown in FIG. 3;

FIG. 5 is an enlarged cross-sectional view in side elevation of a modified telescope portion of the present invention using diffractive lenses;

FIG. 6 is a top plan view of an intraocular implant similar to that shown in FIGS. 3 and 4, but using U-shaped haptics;

FIG. 7 is a side elevational view of the intraocular implant shown in FIG. 6;

FIG. 8 is a cross-sectional view in side elevation of a human eye with an intraocular implant according to a second embodiment of the present invention with an artificial IOL substituted for the natural lens;

FIG. 9 is a cross-sectional view in side elevation of a human eye with an intraocular implant according to a third embodiment of the present invention used with the natural lens;

FIG. 10 is a cross-sectional view in side elevation of a human eye with an intraocular implant according to a fourth embodiment of the present invention;

FIG. 11 is a cross-sectional view in side elevation of a human eye with an intraocular implant according to a fifth embodiment of the present invention;

FIG. 12 is an enlarged cross-sectional view in side elevation of the telescope portion of the intraocular implant of FIG. 11 having a plus and a minus lens therein;

FIG. 13 is an enlarged cross-sectional view in side elevation of alternative telescope portion of the present invention for use with the embodiment of FIG. 11;

FIG. 14 is an enlarged cross-sectional view in side elevation of another alternative telescope portion for use with the embodiment of FIG. 11.

FIG. 15 is a cross-sectional view in side elevation of the embodiment of FIG. 1 further including a contact lens on the cornea;

FIG. 16 is a cross-sectional view in side elevation of the embodiment of FIG. 1 further including an external spectacle; and

FIG. 17 is a top plan view of a bifocal contact lens.

### DETAILED DESCRIPTION OF THE INVENTION

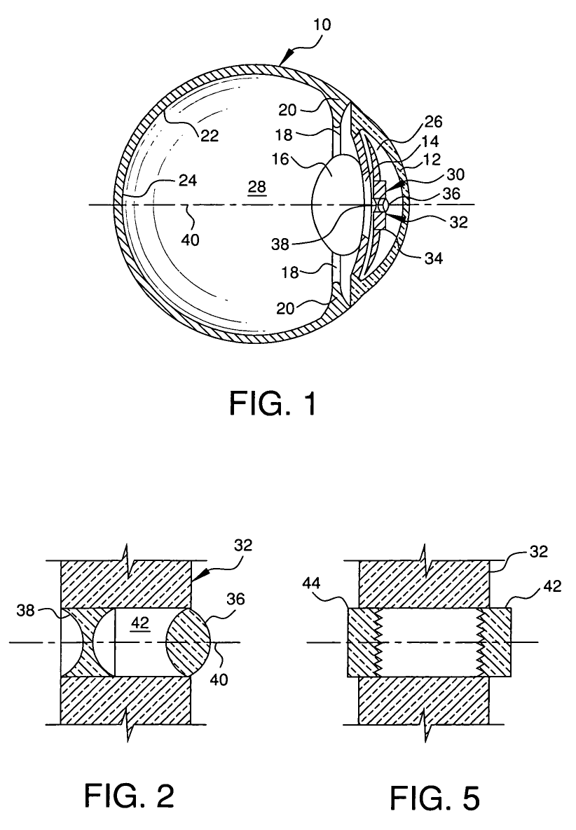

Referring to FIGS. 1-4, an eye 10 includes a cornea 12, iris 14, natural lens 16, zonular ligaments 18, ciliary sulcus 20, retina 22, and macula 24. The natural lens 16, zonular ligaments 18, and ciliary sulcus 20 divide the eye into an anterior chamber 26 and a posterior chamber 28. The macula 24 is located at the center of the retina 22, and is responsible for providing acute vision, such as that necessary for driving or reading. An intraocular telescopic lens implant 30 in accordance with the invention is implanted in the anterior chamber 26 of the eye 10. The intraocular telescopic lens implant 30 has a telescope portion 32 surrounded by a substantially transparent peripheral portion 34.

The telescope portion 32 allows light to pass therethrough and has a bi-convex converging, or plus, lens 36 and a bi-concave diverging, or minus, lens 38. The lenses 36, 38 are aligned along an optical axis 40 to form a Galilean telescope. Preferably, the lenses are about 1-2 mm in diameter. The diverging lens 38 has a refractive index between −30 and −90 diopters, as measured in water. The converging lens 36 has a refractive index between +30 and +80 diopters, as measured in water. The lenses 36, 38 are rigidly received in and fastened as necessary to the wall of a substantially cylindrical aperture 39 formed in the peripheral portion 34 of the implant 30, forming a cavity 42 therebetween. The cavity 42 is preferably vacuum sealed. The use of a vacuum in cavity 42 increases the refractive index, allowing for a smaller telescope. The lenses 36, 38 can be forced-fit or adhered to the aperture 39 so they do not move relative thereto. The lenses 36, 38 are spaced approximately 0.5 to 5 mm apart, depending on their particular optical properties, so that the telescope portion is approximately 0.3 to 5 mm thick.

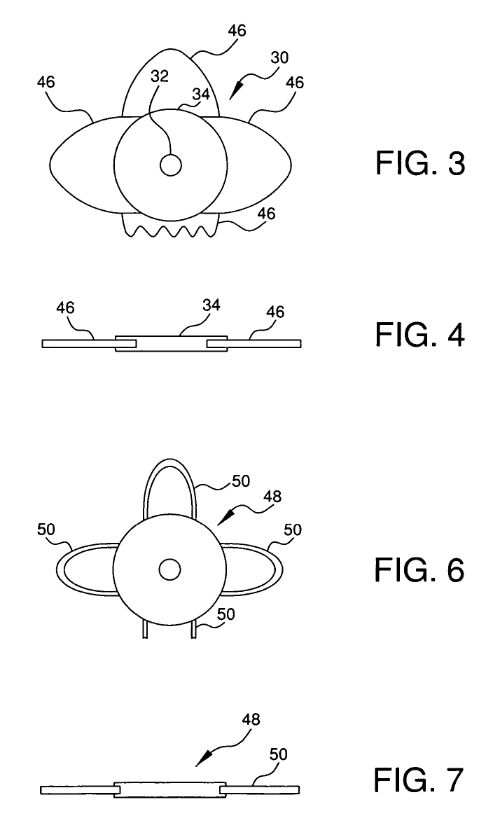

FIGS. 3 and 4 illustrate the intraocular telescopic implant 30 prior to implantation. The substantially circular peripheral portion 34 surrounding or substantially surrounding the telescope portion 32 is made of a biocompatible, transparent, optical material. The peripheral portion has a diameter of approximately 2 to 6.5 mm, and a thickness of approximately 0.05 to 1 mm. The peripheral portion 34 may have refractive powers to correct for refractive errors in the eye, or may have substantially no refractive powers. The peripheral portion 34 may also have varying thickness and refractive power to correct for any astigmatisms in the eye. Further, the peripheral portion 34 can have multiple focal adjustments—i.e., bifocal—to correct for and provide multiple refractive corrections. Arranged around the edge of the peripheral portion 34 are from two to four haptics 46 for fastening the implant in the anterior chamber of the eye. Four haptics are shown in the illustrated embodiment, but any number of haptics may be used. With the haptics, the diameter of the implant is approximately 10-14 mm.

To implant the intraocular telescopic implant in the eye, an incision is made in the eye through the use of a microkeratome, laser, or other suitable surgical device. The implant 30 is folded or rolled up, and inserted into the anterior portion of the eye through the incision. The implant 30 is allowed to unfold or unroll, and the haptics 46 extend into the anterior chamber angle (i.e. the angle formed where the iris and the cornea meet) and fixate the implant into the anterior chamber 26 of the eye 10. Since the implant 30 is foldable, the incision is relatively small. This is beneficial because any incision to the eye can cause astigmatisms in the eye and require varying healing periods. The implant 30 may also be implanted into the posterior chamber, as shown in FIG. 10 and discussed below, or implanted into the capsular bag.

In use, the light rays that enter the eye from the central field of vision are substantially parallel to the axis 40 of the telescopic implant 30. Because they are parallel to the axis of the telescope, the rays enter the telescope and are magnified and projected onto the retina to provide enhanced acute vision for the central field of vision. At the same time, light rays from the peripheral field are unobstructed by the transparent peripheral portion 34 of the lens implant so that the patient retains unrestricted peripheral vision. Furthermore, because the peripheral portion of the implant is transparent, a doctor examining a patient's retina has an unobstructed view of the retina.

The lenses 36, 38 illustrated in FIGS. 1-2 are conventional bi-convex and bi-concave lenses. The conventional lenses are refractive lenses—i.e. they utilize refraction to modify how light propagates through the lenses to change the focal point of the lenses. The lenses in the telescopic implant 30, however, may have any desirable shape or configuration. FIG. 5 illustrates a telescope portion 32 which uses diffractive lenses 42, 44. Diffractive lenses, such as Fresnel lenses, utilize diffraction to modify how light propagates through the lenses to change the focal point of the lenses. Diffractive lenses are advantageous because they are very thin as compared to conventional refractive lenses. Other suitable lenses include those made by ThinOptx, Inc. of Abingdon, Va. ThinOptx, Inc. manufactures intraocular lenses that are approximately 100 microns thick with +/−25 diopters of correction. Further details regarding these lenses are found in U.S. Pat. Nos. 6,666,887 and 6,096,077, which are hereby incorporated by reference in their entirety. When using technology such as this, the telescope portion can be about 2-3 mm, preferably about 2 mm, thick.

The implant 30 illustrated in FIG. 1 uses haptics 46 which affix the implant into the anterior chamber angle. FIGS. 6 and 7 illustrate an implant 48 which uses alternative, substantially U-shaped haptics 50. Upon implantation, the U-shaped haptics 50 overlie the iris and can be clipped to the iris to provide added stability to the implant. One skilled in the art will recognize that although two preferred styles of haptics are specifically disclosed herein, there are a wide variety of known haptics and any suitable haptics, such as J-shaped haptics, can be used with the present invention.

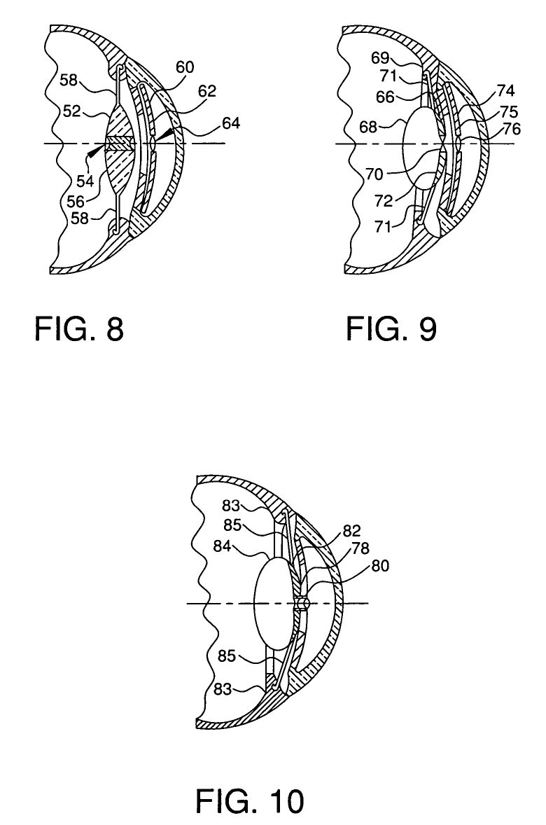

### Embodiment of FIG. 8

FIG. 8 shows a second embodiment of the present invention. In this embodiment, the natural lens of the eye is replaced with an artificial lens 52. The artificial lens 52 has a central portion 54, a peripheral portion 56, and is fastened into the posterior chamber by haptics 58. The peripheral portion 56 of the lens 52 is a generally converging lens, much like the natural lens which it replaces. The central portion 54, however, is a diverging lens with a high negative refractive index. An anterior implant 60 is located in the anterior chamber of the eye. The anterior implant 60 has a transparent peripheral portion 62 and a central portion 64. The central portion 64 is a lens with a high positive refractive index. The anterior implant central portion 64 is aligned with the artificial lens central portion 54, forming a telescope for enhancing low vision. The peripheral portion 62 has the same characteristics as peripheral portion 34 described above regarding the first embodiment of FIGS. 1-4.

### Embodiment of FIG. 9

FIG. 9 illustrates a third embodiment of present invention. In this embodiment, a first intraocular implant 66 is placed immediately adjacent the primary lens 68 and placed in the ciliary sulcus 69 of the posterior chamber by haptics 71. The illustrated primary lens 68 is a natural lens, but may also be an artificial intraocular lens. The central portion 70 of the implant 66 is a lens with a high negative refractive index and is surrounded by a peripheral portion 72, which has the same characteristics as portion 34 described above. A second intraocular implant 74 is placed in the anterior chamber of the eye. The second intraocular implant 74 has a central lens portion 76 with a positive refractive index and a peripheral portion 77 surrounding lens portion 76. The central portions 70, 76 of the two implants 66, 74 are aligned, forming a telescope as discussed above regarding the embodiment of FIGS. 1-4.

### Embodiment of FIG. 10

FIG. 10 shows a fourth embodiment of the present invention. In this embodiment, the intraocular implant 78 has a telescope portion 80 attached to a peripheral portion 82. The peripheral portion 82 is placed directly onto the primary lens 84 and is attached to the ciliary sulcus 83 by haptics 85. The illustrated primary lens is a natural lens, but may also be an artificial intraocular lens. The telescope portion 80, which is constructed in the same manner as previously discussed, extends through the iris. The peripheral portion 82 has the same characteristics as portion 34 described above.

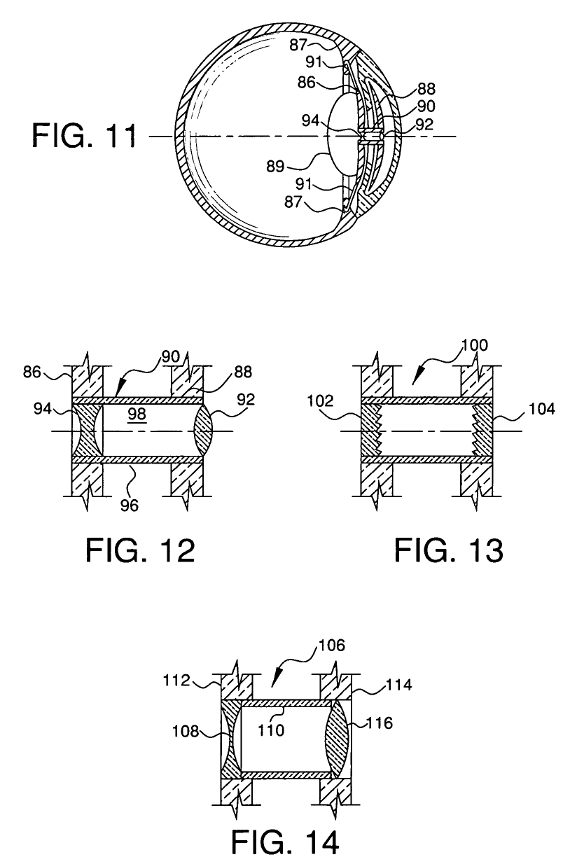

### Embodiment of FIGS. 11 and 12

FIGS. 11 and 12 show a fifth embodiment of the present invention. In this embodiment, a first peripheral portion 86 is located in the posterior chamber of the eye, immediately adjacent the primary lens 89. A second peripheral portion 88 is located in the anterior chamber of the eye. A telescope portion 90 is formed by a converging lens 92, a diverging lens 94, and a tubular canister 96. The tubular canister 96 is rigidly received in circular apertures in the two peripheral portions 86, 88 and rigidly connects the two peripheral portions 86, 88 through the iris. The connection of the canister 96 at both the posterior and anterior chambers of the eye improves the stability of the telescope. The cavity 98 within tubular canister 96 may be vacuum sealed, or may contain air or water. To implant the telescope portion 90 of FIG. 12, the first peripheral portion 86 is inserted into the eye and placed in the sulcus 87 over the primary lens 89 by haptics 91. The illustrated primary lens 89 is a natural lens, but may also be an intraocular lens. The telescope portion 90 is then fastened to the first peripheral portion 86. The second peripheral portion 88 is inserted into the anterior chamber and is fastened to the telescope portion 90. The peripheral portions 86, 88 have the same characteristics as portion 34 described above.

FIGS. 13 and 14 show two additional telescope portions which are suitable for use in the embodiment of FIG. 11. The telescope portion 100 shown in FIG. 13 is similar to the one in FIG. 12, but uses diffractive or Fresnel lenses 102, 104 lenses instead of conventional refractive convex and concave lenses. In the telescope portion 106 shown in FIG. 14, the diverging lens 108 and canister 110 are fastened to the first peripheral portion 112 prior to implantation, and the connected pieces are implanted simultaneously. The second peripheral portion 114 and anterior lens 116 are then implanted, forming the telescope portion in situ. By assembling the telescope portion in this manner, the incision is kept to the smallest possible size.

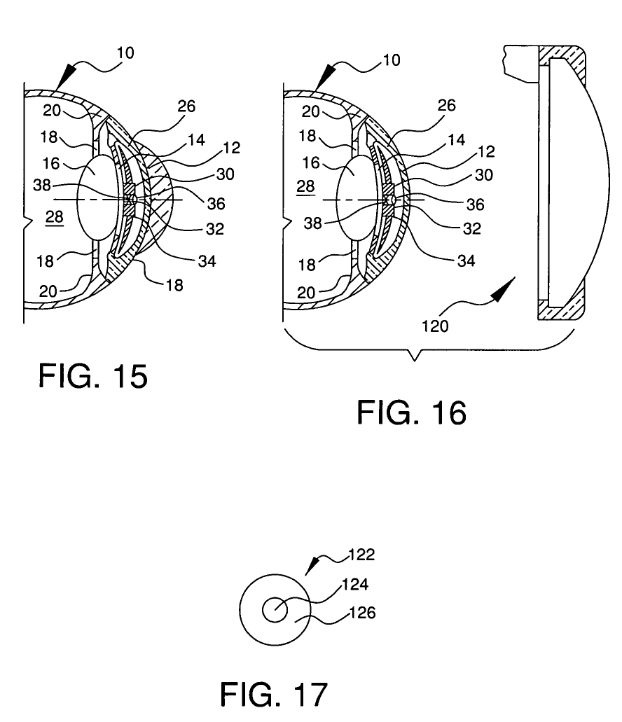

### Embodiment of FIGS. 15-17

Although the invention so far has been described without the use of a supplemental lens outside the eye, it should be understood that the implants can also be used in conjunction with a supplemental lens located outside the eye. FIGS. 15 and 16 illustrate this. In FIG. 15, a supplemental plus contact lens 118 is placed on the cornea 12. In FIG. 16, a supplemental spectacle with two plus lenses 120 is placed in the visual path. In both cases, the lenses 118, 120 have a positive refractive index. The use of supplemental lenses outside the eye allows for smaller implants inside the eye. Further, the use of supplemental lenses allows the construction and operation of the implants to be tailored to particular patients' desires. For instance, many individuals have a preferable reading distance (typically between 20 and 50 cm away from the eye) and a supplemental lens allows the focal distance to be tailored to coincide with an individual's preferred reading distance. The supplemental lenses themselves can be bifocal. FIG. 17 illustrates a contact lens 122. The central 2-5 mm portion 124 of the contact lens 122 provide refractive correction for near vision. The peripheral portion 126 provides refractive correction for far vision.

While various embodiments have been chosen to illustrate the invention, it will be understood by those skilled in the art that various changes and modifications can be made therein without departing from the scope of the invention as defined in the appended claims.

1. An intraocular lens implant for implantation in an eye having an anterior chamber and a posterior chamber, comprising:
a telescope portion having a converging lens and a diverging lens;
a first peripheral portion coupled to the outside of said telescope portion, said first peripheral portion having refractive powers to correct for refractive errors in the eye; and
a first set of haptics for fastening said first peripheral portion in said eye.

2. An intraocular lens implant according to claim 1, wherein
said first peripheral portion is adapted to be implanted into said anterior chamber of said eye.

3. An intraocular lens implant according to claim 2, further including
a second peripheral portion coupled to the outside of said telescope portion, said second peripheral portion being substantially transparent; and
a second set of haptics for fastening said second peripheral portion in said posterior chamber of said eye.

4. An intraocular lens implant according to claim 1, wherein
said converging and diverging lenses are separated by a vacuum.

5. An intraocular lens implant according to claim 1, wherein
said converging and diverging lenses are refractive lenses.

6. An intraocular lens implant according to claim 1, wherein
said converging and diverging lenses are diffractive lenses.

7. An intraocular lens implant according to claim 6, wherein
said diffractive lenses are Fresnel lenses.

8. An intraocular lens implant according to claim 1, wherein
said first peripheral portion is toric to correct an astigmatism in said eye.

9. An intraocular lens implant according to claim 1, further comprising
a supplemental lens for use outside the eye.

10. An intraocular lens implant kit for implantation in an eye, comprising:
a first intraocular lens for replacing a natural lens, said first intraocular lens having a central portion and a peripheral portion, said central portion comprising a lens with a negative refractive index, said peripheral portion having refractive powers to correct for refractive errors in the eye;
a second intraocular lens adapted to be placed in said anterior chamber of said eye, said second intraocular lens having a central portion comprising a lens with a positive refractive index.

11. An intraocular lens implant kit according to claim 10, wherein
said central portion of said first intraocular lens is a diffractive lens.

12. An intraocular lens implant kit according to claim 10, further comprising
a supplemental lens adapted to be located outside the eye and cooperate with the first and second intraocular lenses.

13. An intraocular lens implant according to claim 12, wherein
said supplemental lens is bifocal.

14. An intraocular lens implant kit for implantation in an eye having a primary lens in a posterior chamber and an anterior chamber, comprising:
a first intraocular lens adapted to be placed on a surface of said primary lens of said eye, said lens having a peripheral portion and a central portion, said central portion having a negative refractive index, said peripheral portion having refractive powers to correct for refractive errors in the eye;
a second intraocular lens adapted to be placed in said anterior chamber of said eye, said second intraocular lens having a central portion with a positive refractive index.

15. An intraocular lens implant kit according to claim 14, wherein
said primary lens is a natural lens.

16. An intraocular lens implant kit according to claim 14, wherein
said primary lens is an artificial lens.

17. An intraocular lens implant kit according to claim 14, wherein
said central portion of said first intraocular lens is a diffractive lens.

18. An intraocular lens implant kit according to claim 14, wherein
said central portion of said second intraocular lens is a diffractive lens.

19. An intraocular lens implant kit according to claim 14, further comprising
a supplemental lens adapted to be located outside the eye and cooperate with the first and second intraocular lens.

20. An intraocular lens implant kit according to claim 19, wherein
said supplemental lens is bifocal.

21. An intraocular lens implant for implantation in an eye having a primary lens, comprising:
an intraocular lens adapted to be placed on a surface of said primary lens of said eye, said intraocular lens having a peripheral portion and a telescope portion, said telescope portion having a first lens and a second lens separated by a vacuum.

22. An intraocular lens implant according to claim 21, wherein
said primary lens is a natural lens.

23. An intraocular lens implant according to claim 21, wherein
said primary lens is an artificial lens.

24. An intraocular lens implant according to claim 21, wherein
said first lens is a converging lens; and
said second lens is a diverging lens.

25. An intraocular lens implant according to claim 21, wherein
said first and second lenses are refractive lenses.

26. An intraocular lens implant according to claim 21, wherein
said first and second lenses are diffractive lenses.

27. An intraocular lens implant according to claim 21, further comprising
a supplemental lens adapted to be located outside the eye and cooperate with said intraocular lens.

28. An intraocular lens implant according to claim 21, wherein
said supplemental lens is bifocal.

29. An intraocular lens implant for implantation in an eye having an anterior chamber and a posterior chamber, comprising:
a first intraocular lens portion adapted to be implanted in said posterior chamber of an eye;
a second intraocular lens portion adapted to be implanted in said anterior chamber of an eye;
a telescope portion connecting said first intraocular lens portion and said second intraocular lens portion, said telescope portion having a converging lens and a diverging lens aligned along an optical axis.

30. An intraocular lens according to claim 29, wherein
said converging and diverging lenses are separated by a vacuum.

31. An intraocular lens according to claim 29, wherein
said converging and diverging lenses are refractive lenses.

32. An intraocular lens according to claim 29, wherein
said converging and diverging lenses are diffractive lenses.

33. An intraocular lens implant kit according to claim 29, further comprising
a supplemental lens adapted to be located outside the eye.

34. A method for correcting low vision in an eye having an anterior chamber and a posterior chamber, comprising the steps of:
inserting a first intraocular lens portion into the posterior chamber of the eye;
attaching a telescope having a converging and a diverging lens to the first intraocular lens portion;
inserting a second intraocular lens portion into the anterior chamber of the eye; and
attaching the second intraocular lens portion to the telescope
